(19) United States
(12) Patent Application Publication
(10) Pub. No.: US 2026/0053476 A1
(43) Pub. Date: Feb. 26, 2026

(54) Bladder Management Device and Method

(71) Applicants: Brian J. Awbrey, Lexington, MA (US); David E. Flinchbaugh, Orlando, FL (US); Allan F. Anderson, Flagler Beach, FL (US); Michael A. Rawers, Bonita Springs, FL (US); Paul Golis, Sanford, FL (US)

(72) Inventors: Brian J. Awbrey, Lexington, MA (US); David E. Flinchbaugh, Orlando, FL (US); Allan F. Anderson, Flagler Beach, FL (US); Michael A. Rawers, Bonita Springs, FL (US); Paul Golis, Sanford, FL (US)

(21) Appl. No.: 19/311,944

(22) Filed: Aug. 27, 2025

Related U.S. Application Data
(63) Continuation-in-part of application No. 18/812,921, filed on Aug. 22, 2024, now Pat. No. 12,419,732.

(51) Int. Cl. A61B 10/00; A61B 5/00; A61B 5/20

(52) U.S. Cl. CPC A61B 10/007; A61B 5/0022; A61B 5/202; A61B 5/207; A61B 5/7264; A61B 5/742; A61B 2560/0214

(57) ABSTRACT

The present disclosure provides a bladder management device that stands as a natural cure device for a major medical problem and a notorious killer. The bladder management device may include a hard, durable body with an inflow portion and an outflow portion. In some implementations, the bladder management device may include a hollow body with a sample port and over-pressurization port and at least two magnets. In some aspects, the bladder management device may accept the influx of drainage fluid from a user. In some implementations, the bladder management device may include one or more diagnostic ports, one or more diagnostic caps, and one or more modular diagnostic pods. In some implementations, the diagnostic pods may send or receive point of care diagnostic information in real time to external devices or mobile applications with AI software for analysis and EHR reporting.

### CROSS-REFERENCE TO RELATED APPLICATIONS

This application claims priority to and the full benefit of U.S. Nonprovisional patent application Ser. No. 18/812,921 (filed Aug. 22, 2024, and titled “BLADDER MANAGEMENT DEVICE AND METHOD”), the entire contents of which are incorporated in this application by reference.

### BACKGROUND

For thousands of years, urinary catheters have been used to gain access and subsequently drain urine from within the bladder to the outside of the body into a drainage bag. Catheters are routinely used to treat a wide variety of patient healthcare issues including bladder problems, urinary sphincter and bladder nerve regulation problems, urinary retention problems, neurogenic bladder and as a temporary solution during, and following, difficult surgical procedures, trauma, ICU admissions or other serious illnesses.

While the catheter has incredible medical benefits, there are a gamut of dangerous side effects and serious risks of morbidity and mortality associated with their use: representing a major medical dilemma. Some of these risks include catheter biofilms, catheter blockage, catheter-associated urinary tract infections (CAUTIs), urethral trauma, bladder spasms, hematuria, and catheter leakage. Even the most advanced modern-day catheters are unable to avoid many of these issues, especially UTIs. CAUTIs occur due to incomplete bladder drainage and due to the loss of the physiologic advantage of bladder cycling: whereby bacteria seed and replicate within the urinary tract due to biofilms, catheter balloon urinary stagnation, encrustation of urine struvite crystals and the chronic catheter foreign body contamination effect. Despite the notorious and excessive infection risk of 3-10% per day, compounding for each day of catheter use, medical professionals must employ the use of a urinary catheter as a life and organ-saving intervention for more than 15% of all hospitalized patients.

These drawbacks to modern day indwelling urinary catheters introduce serious iatrogenic risk of CAUTI, antibiotic resistance and dangerous morbidity/mortality to patients who are often already dealing with serious medical conditions. A further major disadvantage and obstacle to modern day indwelling urinary catheter use is the billions of dollars of annual healthcare costs wasted to treat resultant CAUTI. If there was a catheter, or catheter adapter, that eliminated these drawbacks, eliminated the high incidence for UTIs without additional antibiotics and the other 13 catheter-induced issues, it would greatly elevate the level of catheter care, case the burden of healthcare professionals and reduce the cost of care of these patients. Specifically, mimicking physiologic bladder cycling with an automatic external artificial urinary sphincter prevents CAUTI and bacterial overgrowth of catheter biofilms without need for expensive and potentially harmful antibiotics.

Standardized modern efforts to decrease CAUTI are the SHEA/IDSA/APIC Practice Recommendations of Strategies to Prevent CAUTI in 2014 (updated in 2022): consisting primarily of restricting catheter use, to use non-catheter urinary management supplies (i.e. diapers), ensure that only trained HCPs insert urinary catheters under aseptic conditions, routine use of antimicrobial/antiseptic coated catheters, routine use of oral and parenteral prophylactic antibiotics, and catheter irrigation as a strategy to prevent infection. While heavily monitoring a patient treated with a urinary catheter and limiting catheter access may achieve some results, a catheter adapter that appropriately self-regulates the urinary drainage of a patient with an artificial intelligence urinary bladder management diagnostic device and external prosthetic urinary sphincter solves this major medical dilemma without antibiotics.

### SUMMARY OF THE DISCLOSURE

What is needed is a bladder management device that improves the urinary fluid drainage process while reducing or eliminating unnecessary risk and complication by mimicking physiologic bladder cycling. The invention is a small and inexpensive device that supplants the need for expensive and potentially harmful antibiotics, and prevents urinary tract infections without the use of antibiotics, for millions of urinary catheter users in the US. A bladder management system that is self-scaling, wherein the fluid volume of an individual may increase or decrease the pressure placed on at least two magnets, wherein a certain threshold separates the at least one slidable or articulating or fully movable magnet from the at least one fixed magnet despite their magnetic attraction, which eliminates the majority of negative urine drainage side effects experienced by urinary catheter users. The described AI bladder management diagnostic prosthetic sphincter system is automatically cycling, the base device perpetually operates without an external energy force and it is fully self-energized by fluid dynamics.

In some embodiments, a bladder management device may include a hard, durable body with a hollow chamber. In some aspects, the bladder management device may include an inflow portion, wherein the inflow portion may be configured to accept the influx of drainage fluids from at least one user. In some aspects, the bladder management device may include an outflow portion comprised of distally oriented barbed fins, wherein the outflow portion may be configured to expel fluids to at least one external source. In some implementations, a sample port may be configured to enable the hollow chamber to collect a sterile fluid specimen. In some aspects, an over-pressurization port safety relief valve to ensure failsafe release of excess pressure in the hollow chamber. In some implementations, the bladder management device may include at least two magnets including at least one fixed magnet and one slidable or articulating or fully moveable magnet, wherein the at least one slidable or articulating or fully moveable magnet moves when a certain predetermined threshold of pressure may be experienced.

In some embodiments, the bladder management device may include one or more diagnostic ports protruding from the exterior surface of the hollow chamber, wherein the one or more diagnostic ports may include a pathway into the hollow chamber. In some implementations, the bladder management device may include one or more modular diagnostic pods which are sterilely and detachably connectable to the one or more diagnostic ports, wherein the one or more modular diagnostic pods may be configured to draw in fluids from the hollow chamber. In some aspects, the one or more modular diagnostic pods may be configured to perform chemical testing, urometric testing and diagnostic testing on the drainage fluids before releasing the fluids back into the hollow chamber.

A number of embodiments of the present disclosure will be described. While this specification contains many specific implementation details, these should not be construed as limitations on the scope of any disclosures or of what may be claimed, but rather as descriptions of features specific to particular embodiments of the present disclosure. It is understood to those skilled in the art that variations, modifications, and alterations may be apparent. It will be understood that various modifications may be made without departing from the spirit and scope of the disclosure.

### BRIEF DESCRIPTION OF THE DRAWINGS

The accompanying drawings that are incorporated in and constitute a part of this specification illustrate several embodiments of the disclosure and, together with the description, serve to explain the principles of the disclosure:

FIG. 1 illustrates a bladder management device, according to some embodiments of the present disclosure.

FIG. 2 illustrates a bladder management device with a drainage bag, according to some embodiments of the present disclosure.

FIG. 3 illustrates a bladder management device with a shell protector, according to some embodiments of the present disclosure.

FIG. 4 illustrates a bladder management device with a microchip, according to some embodiments of the present disclosure.

FIG. 5 illustrates a bladder management device with a microchip, according to some embodiments of the present disclosure.

FIG. 6 illustrates a bladder management device with a sample port and an over-pressurization port, according to some embodiments of the present disclosure.

FIG. 7 illustrates a flow chart illustrating the sequential functions of a self-sealing and automatically and perpetually self-energized bladder management prosthetic urinary sphincter device.

FIG. 8 illustrates a bladder management device with one or more diagnostic ports, according to some embodiments of the present disclosure.

FIG. 9 illustrates a bladder management device with one or more modular diagnostic ports, according to some embodiments of the present disclosure.

FIG. 10 illustrates a bladder management device with one or more modular diagnostic ports, according to some embodiments of the present disclosure.

FIG. 11 illustrates a bladder management system with a bladder management platform, according to some embodiments of the present disclosure.

FIG. 12 illustrates a method for bladder management, according to some embodiments of the present disclosure.

The Figures are not necessarily drawn to scale, as their dimensions can be varied considerably without departing from the scope of the present disclosure.

### DETAILED DESCRIPTION

The present disclosure provides generally for a bladder management device. In some aspects, the urine control flow device may be unidirectional, wherein a sample port may allow the sterile withdrawal of urine samples and an over-pressurization port as a fail-safe safety relief valve to ensure failsafe function. According to the present disclosure, a bladder management device may drain the influx of urine, wherein co-acting magnets may control the flow of urine to automatically activate the device to completely empty the bladder in a full cycle to prevent catheter biofilms, CAUTI, loss of bladder muscle strength, infection and sepsis, and bladder damage issues from occurring.

In the following sections, detailed descriptions of examples and methods of the disclosure will be given. The description of both preferred and alternative examples, though thorough, are exemplary only, and it is understood to those skilled in the art that variations, modifications, and alterations may be apparent. It is therefore to be understood that the examples do not limit the broadness of the aspects of the underlying disclosure as defined by the claims.

### Glossary

Bladder management device: as used herein refers to a device configured to drain and then physiologically cycle the influx of urine from a catheter user and act as an automatic self-energizing external prosthetic urinary sphincter or explantable (i.e. removable) or implantable prosthetic urinary sphincter.
Catheter-Associated Urinary Tract Infection (CAUTI): as used herein refers to an iatrogenic urinary tract infection with a daily patient risk rate of 7-10%, which is associated with severe morbidity, significant mortality and wasteful excess healthcare costs.
Sample port and Over-Pressurization port: as used herein refers to a feature of the bladder management device that has the dual purpose of providing sterile access to the influx of urine fluid specimens and, also, for the purpose of expelling excess fluid pressure experienced as a safety relief valve to ensure failsafe function by the bladder management device.
Electronic Health Record (EHR): as used herein refers to a digital health record or health record software, which includes medical monitoring and reporting.
Distal oriented barbed fins: as used herein refers to protrusions located on the outflow portion of the bladder management device that are configured to secure the bladder management device to an external tubing.
Artificial Intelligence Software (AI): as used herein refers to software which is able to accumulate, self-analyze and report significant changes in the digital health record directly to the dashboard of medical care professionals in real-time based upon established parameters.
Shell Protector: as used herein refers to an accessory to the bladder management device that provides protection against electromagnetic interference and other types of electronic threats.
Inflow Portion: as used herein refers to the upstream section of the bladder management device where fluids are drained from a catheter user.
Outflow Portion: as used herein refers to the downstream section of the bladder management device with distally oriented fins where the bladder management device may connect to an external device to drain and deposit fluids received from the inflow portion.
Diagnostic Ports: as used herein refers to a component of the bladder management device configured to allow direct access of the drainage fluids within the hollow chamber of the device. In some aspects, the diagnostic ports may be configured to connect to diagnostic caps configured to seal off access or modular diagnostic pods configured to draw fluids from the bladder management device.
Modular Diagnostic Pods: as used herein refers to a component of the bladder management device that may draw sterile fluid for chemical analysis. In some aspects, the modular diagnostic pods may be detachably connectable to the bladder management device through diagnostic ports located on the exterior of the bladder management device.
Microfluidic Chip: as used herein refers to an electronic component that may be included in the modular diagnostic pods and may be a stimulus responsive wireless sensor and transmitter lab on a chip device for measuring microdroplets, wherein the microfluidic chip may be configured to analyze a low volume of fluid for chemical analysis, biological assays, and medical diagnostics, as non-limiting examples.
Bladder Management Platform: as used herein refers to a software application electronically coupled to a bladder management device, with or without AI integration, wherein the bladder management platform may be configured to organize and display the results of the bladder management device, including the modular diagnostic pods. In some aspects, the bladder management platform may be used to control the functionality of the bladder management device.

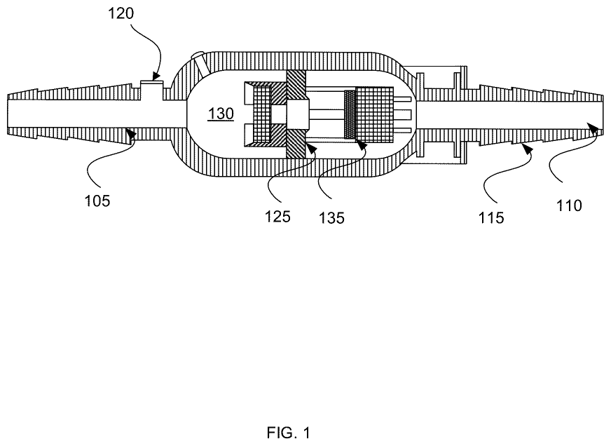

Referring now to FIG. 1, a bladder management device, according to some embodiments of the present disclosure, is illustrated. In some aspects, the bladder management device may include a hard, durable body with an inflow portion 105 and an outflow portion 110. In some aspects, the hard, durable body may be transparent. In some implementations, the hard, durable body may include a hollow chamber 130. In some embodiments, the hollow chamber 130 may include a combination sample port and over-pressurization port 120 and at least two magnets 125, 135. In some embodiments, the sample port and over-pressurization port 120 may be located separately, wherein a sample port and an over-pressurization port are two different accessories to the bladder management device.

In some aspects, the bladder management device may be configured to accept the influx of drainage fluids from the inflow portion 105 through the hollow chamber 130 and send the drainage fluids out through the outflow portion 110. In some implementations, the drainage fluids may be drawn by the pressure induced by at least one external fluid source, wherein the pressure may induce the at least two magnets to move in such a way that allows the drainage fluid to move through the hollow chamber 130 from the inflow portion 105 to the outflow portion 110 of the device. In some aspects, the at least one external fluid source may be a user, wherein the user may be a patient. In some implementations, the user may be domestic or livestock animal or other type of living creature, as non-limiting examples.

In some embodiments, the outflow portion 110 may include a plurality of distally oriented barbed fins 115, wherein the plurality of distally oriented barbed fins 115 may be configured to connect the bladder management system to an external device. In some implementations, the sample port and over-pressurization port 120 may be upstream from at least two magnets that may be configured to sense the pressure of urine build up in a user's bladder, wherein the increase of pressure may separate the at least two magnets from one another to enable the urine to move through the bladder management device. In some aspects, the at least two magnets may include at least one fixed magnet 125 and one slidable or articulating or freely moving magnet 135, wherein the at least one fixed magnet 125 may be unmovable and the at least one slidable or articulating or fully movable magnet 135 may move when a certain predetermined threshold of pressure may be experienced.

In some aspects, the bladder management device may be self-sealing, wherein the increase or decrease of bladder fluid pressure in an individual may increase or decrease the pressure placed on the at least two magnets, wherein the certain pressure threshold separates the at least one slidable or articulating or freely moveable magnet 135 from the at least one fixed magnet 125 despite their magnetic attraction. In some implementations, the decrease in pressure may be outweighed by the force of attraction between the at least two magnets, wherein the at least two magnets may connect, thereby closing off a path through the bladder management device. In some aspects, the closed path of the bladder management device prevents the drainage of any fluids from the prosthetic sphincter despite further influx of user fluid.

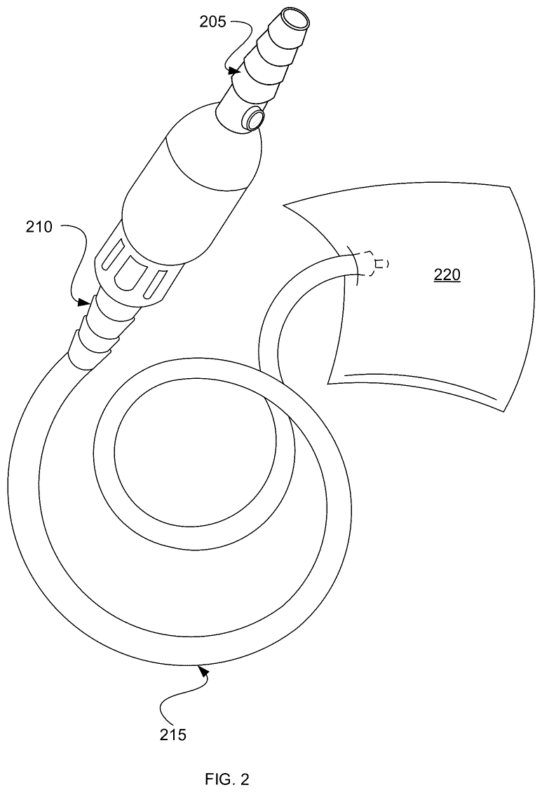

Referring now to FIG. 2, a bladder management device with a drainage bag, according to some embodiments of the present disclosure, is illustrated. In some embodiments, the bladder management device may include an inflow portion 205 and an outflow portion 210. In some aspects, the outflow portion 210 may be connected to a tubing 215, wherein the tubing 215 may be attached to the drainage bag 220. By way of example and not limitation, fluids may travel from an individual, through the bladder management device and into the drainage bag 220, wherein the drainage bag 220 may be configured to store the drained urine.

In some implementations, the bladder management device may include at least two magnets. In some aspects, the increase of pressure may be configured to separate the at least two magnets to open the passageway of the bladder management device so that it may move into the drainage bag. In some embodiments, the outflow portion 210 may include a plurality of distally oriented barbed fins, wherein the plurality of distally oriented barbed fins may be placed inside the internal cavity of the tubing. In some aspects, placing the plurality of distally oriented barbed fins into the tubing may secure the bladder management device with the drainage bag 220.

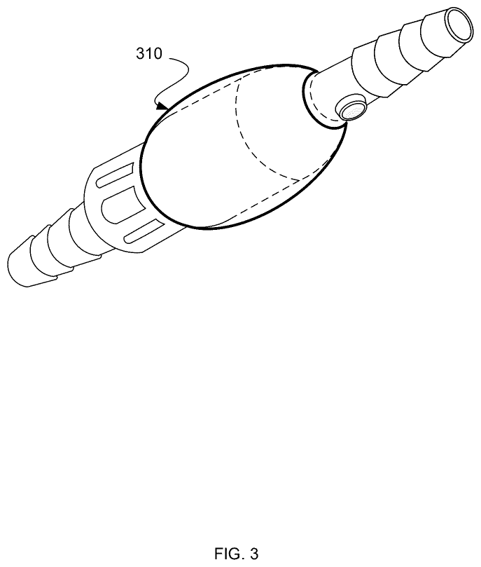

Referring now to FIG. 3, a bladder management device with a shell protector, according to some embodiments of the present disclosure, is illustrated. In some embodiments, the bladder management device may include a shell protector 310. In some aspects, the shell protector 310 may be configured to envelope the body of the bladder management device. In some implementations, the shell protector 310 may provide electro-magnetic pulse protection to prevent this bladder management device and critical medical devices from failing in the presence of foreign threat. In some aspects, the shell protector 310 may cover at least a portion of the bladder management device.

In some embodiments, the shell protector 310 may consist of materials that interfere with electromagnetic fields. By way of example and not limitation, the materials that make up the shell protector may include aluminum, carbon fiber, chromium, copper, or a nickel-iron soft ferromagnetic alloy. In some aspects, the shell protector 310 may be configured as a sleeve. In some implementations, the shell protector 310 may be attachable and removable, wherein the shell protector 310 may be placed over the bladder management device when the bladder management device may be in use. In some aspects, the shell protector 310 may be glued, press fit, elastically attached or locked onto the bladder management device, as a non-limiting example.

In some embodiments, the shell protector 310 may provide passive and active protection. In some aspects, the shell protectors' 310 passive protection may include radio frequency identification blocking or blocking shields that protect any enclosed electromagnetic components so that they are safe and unreadable from external electrical fields such as an MRI signal or bedside cardiac monitor, as a non-limiting example. In some implementations, the shell protector 310 may include active protection. In some aspects, the active shell protector 310 may be configured to emit a predetermined threshold of radio frequencies to disrupt and interfere with signals that may have an impact on the functionality of the bladder management device.

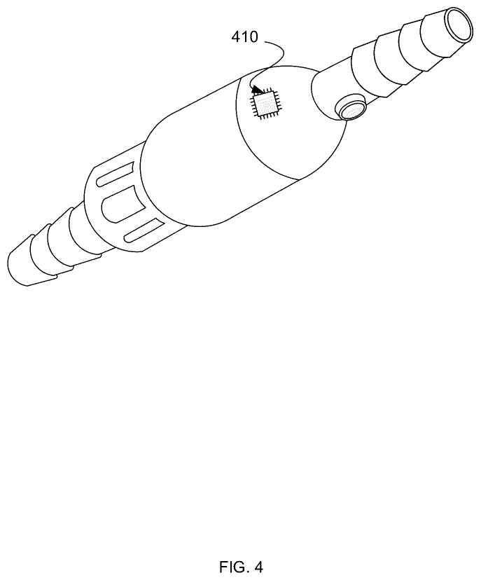

Referring now to FIG. 4, a bladder management device with a microchip, according to some embodiments of the present disclosure, is illustrated. In some embodiments, the body of the bladder management device may include a microchip 410 on its external surface. In some aspects, the microchip 410 may be a small electronic device configured to perform a variety of electronic functions. In some implementations, the microchip 410 may be configured to provide active pulse protection or to send signals to an external device, as non-limiting examples.

In some implementations, the microchip 410 may provide active pulse protection for the bladder management device. In some aspects, active pulse protection may eliminate any risk of electromagnetic pulse malfunction to ensure the bladder management device is working properly. In some embodiments, the active pulse protection may be configured to send out radio frequency waves that may prevent unauthorized access to the bladder management device or any other effect from disrupting the device.

In some implementations, the microchip 410 may include a battery power source. In some aspects, the battery power source may be replaceable or rechargeable, as non-limiting examples. In some implementations, the replaceable battery source may include an accessible battery that may be attached and removed from the microchip 410. In some aspects, the rechargeable battery may include a rechargeable portion that may automatically recharge or may be charged through an external energy device or direct-wired to a low voltage power source.

In some embodiments, the microchip 410 may be communicatively coupled with at least one external device. In some aspects, the microchip 410 may be configured to send and receive signals from the at least one external device. By way of example and not limitation, the at least one external device may include a computer device, mobile device, or digital system. In some implementations, the at least one external device may be configured to analyze, process, and display the status and results of the bladder management device, wherein signals the microchip 410 receives from the bladder management device may be sent from the microchip 410 to the at least one external device.

In some implementations, the functioning of the bladder management device may be analyzed and controlled by the at least one external device. In some aspects, the at least one external device may activate the bladder management device to open or close upon command to drain fluids from a user. In some embodiments, the at least one external device may analyze the results of the upstream influx of drainage fluids within the upstream portion of the bladder management device, wherein details of the fluid and the bladder management device's user's health status may be, variously, digitally or visually or AI digitally revealed to bedside or physically-distant monitoring medical professionals, as well as permanently stored within the electronic medical record. As a non-limiting example, the upstream urine diagnostic analysis device and capabilities would be for point-of-service and instant digital measurement, EHR reporting and AI diagnostic analysis of: cell type and specificity, bacteria volume and identification, routine urine chemistry values, bacteria quantity and identification, metabolites of sepsis, color, pH, specific gravity, temperature, volume, real-time pressure metrics, cardiac enzymes, glucose, instant illicit drug detection, excreted medication toxic level detection, bladder metrics flow analysis, metabolites of hormones/disease states/cancers or pathologic enzymes precise for disease states.

In some embodiments, the microchip 410 and the at least one external device may be coupled to communicate, by any of several methods, directly with the Electronic Health Record (EHR), external monitors or include an artificial intelligence (AI) software. In some aspects, the AI software may be configured to receive and analyze signal from the bladder management device. In some implementations, the AI software may be configured to control the function of the bladder management device, wherein the AI software may prompt the bladder management device to drain, or fully stop the drainage of fluids from a user. In some embodiments, the AI software may be configured to determine the presence of any improving or untoward issues by instantly analyzing the urine of a user digitally and remotely. By way of example and not limitation, these issues may include catheter blockage, urinary tract infections (UTIs), urethral trauma, bladder spasms, hematuria, and catheter leakage.

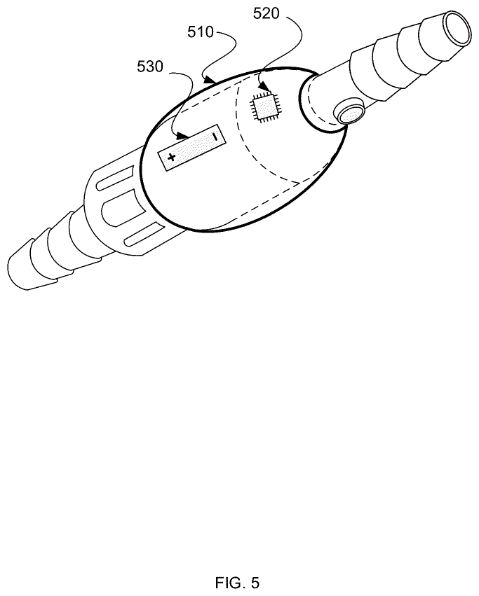

Referring now to FIG. 5, a bladder management device with a microchip, according to some embodiments of the present disclosure, is illustrated. In some embodiments, the bladder management device may include a shell protector 510 and a sensor or microchip 520. In some aspects, the exterior surface of the shell protector 510 may include the microchip 520. In some implementations, the microchip 520 may be removable from the shell protector 510, wherein the microchip 520 may be replaced if necessary. In some aspects, the microchip 520 may access the bladder management device through the shell protector 510, wherein the microchip 520 may be configured to receive signals from the bladder management device to send the signals to an external device. In some embodiments, the microchip 520 may connect directly to the battery or any non-limiting local power source and/or at least one device to analyze details of the upstream fluid.

In some embodiments, the microchip 520 may include a battery source 530, wherein the battery source 530 may be replaceable or rechargeable, as non-limiting examples. In some aspects, the microchip 520 may include analytic sensors. In some implementations, the sensors may be configured to receive-detect neural signals actuating the magnets to open or close upon neural command, as a non-limiting example. In some aspects, the microchip 520 may communicate with the bladder management device, wherein the microchip 520 may send and receive signals to and from the bladder management device. In some aspects, the microchip 520 may communicate with at least one chemical analyzer, cell analyzer, fluid volume analyzer or urometric pressure gauge, as a non-limiting example.

By way of example and not limitation, the coupled microchips 520 of the bladder management device may communicate with one another to optimize, or customize, the fluid drainage process. In some implementations, the shell protector 510 may protect the microchip 520 from digital interference and electromagnetic pulses, as non-limiting examples. In some embodiments, the shell protector 510 may be digitally coupled to the microchip 520, wherein the microchip 520 may receive and send signals from the bladder management device and shell protector 510. In some implementations, the microchip 520 may be communicatively coupled with at least one external device, wherein the at least one external device may be configured to send and receive signals from the microchip 520.

In some aspects, the at least one external device may include a computer device or mobile device, as non-limiting examples. In some embodiments, the at least one external device may be configured to analyze, process, and display the information received by the signals of the sensor or microchip 520 and interact directly with a user or HCP controlled application. In some aspects, the at least one external device may display the information on a digital display. In some embodiments, the at least one external device may be configured to control the shell protector 510 and the bladder management device, wherein the microchip 520 may receive signals from the at least one external device and send directions to the shell protector 510 and the bladder management device.

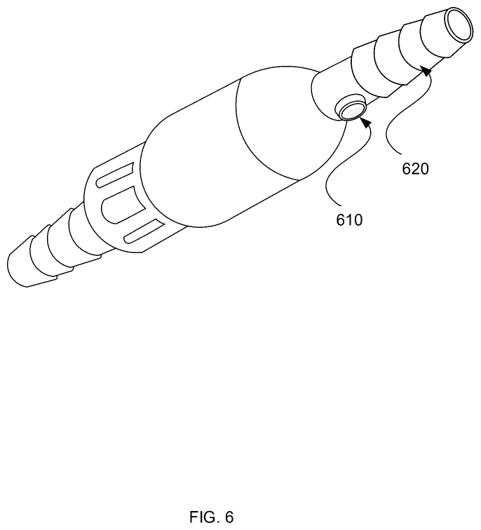

Referring now to FIG. 6, a bladder management device with a sample port and over-pressurization port fail-safe relief valve, according to some embodiments of the present disclosure, is illustrated. In some embodiments, the sample port and over-pressurization port 610 may prevent the bladder management device from failure. In some implementations, the sample port and over-pressurization port 610 may be located on the bladder management device at the inflow portion 620, wherein the sample port and over-pressurization port 610 is located before the distally oriented barbed fins. In some aspects, the sample port and over-pressurization port 610 may be externally or internally mounted on the bladder management device. In some embodiments, the sample port and over-pressurization port 610 fail-safe relief valve may be a disc-like elastomeric diaphragm, wherein the diaphragm may include at least one circumferential biased tooth. In some aspects, the circumferential biased tooth may include a horizontal lower surface and an upwardly angled upper surface.

In some implementations, the sample port and over-pressurization port 610 may serve as an over-pressurization port, wherein the over-pressurization port may be used as a pressure relief fail-safe valve, wherein when a predetermined threshold of pressure may be experienced, the valve may open to release the pressure. By way of example and not limitation, the predetermined threshold of pressure may be any supraphysiologic bladder pressure greater than 40 cm of fluids. In some aspects, the over-pressurization port may prevent excess pressurization of the upstream fluid path, wherein the over pressurization of the fluid path may be caused by pathway obstruction, magnet failure, patient positional obstruction, intentional obstruction, bacterial obstruction, or debris buildup, as non-limiting examples.

In some embodiments, the sample port and over-pressurization port 610 may be used to sample a sterile urine specimen under direct visualization in the device. In some aspects, the sample port and over-pressurization port 610 may allow the bladder management device to collect sterile fluid specimen without breach of the sterile enclosed system. In some embodiments, the sample port and over-pressurization port 610 may enable the device to catch the freshest fluids as required by medical-grade regulations. In some implementations, the sample port and over-pressurization port 610 may be comprised of a disc-like elastomeric or plastic material that may be pierceable by a conventional, sterile syringe needle, wherein the sample port and over-pressurization port 610 may be capable of sterilely and fully rescaling itself after the needle may be withdrawn.

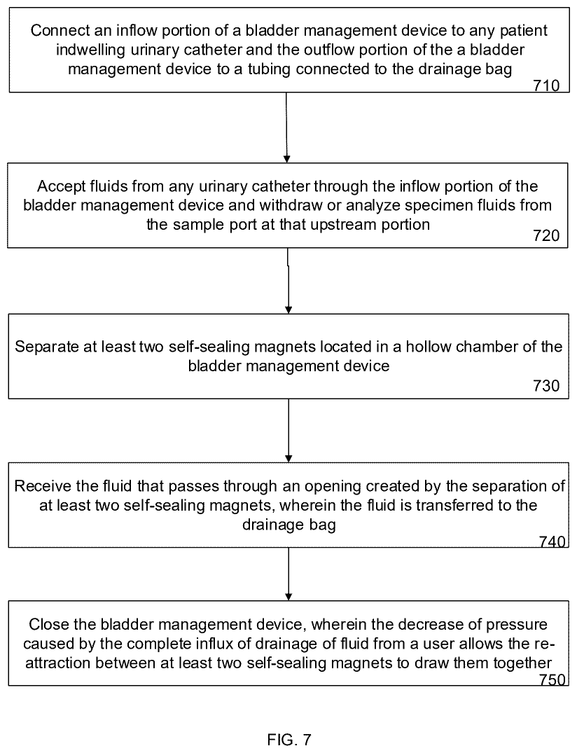

Referring now to FIG. 7, a method for a bladder management system, according to some embodiments of the present disclosure, is illustrated. At 710, the method includes connecting an inflow portion of a bladder management device to any patient indwelling urinary catheter and the outflow portion of a bladder management device to a tubing connected to a drainage bag. In some aspects, at 720 may further include the influx of drainage fluids from a user through the inflow portion of the bladder management device and withdrawing or analyzing specimen fluids from the sample port at that upstream portion. At 730, the method may include separating at least two self-sealing magnets located in a hollow chamber of the bladder management device, wherein the increase in certain and safe fluid pressure within the bladder of the user forces an at least one slidable or articulating or fully movable magnet to move away from the at least one fixed magnet.

At 740, the method may involve receiving the fluid that passes through an opening created by the separation of at least two self-sealing magnets, wherein the fluid passes through an outflow portion of the bladder management device and into a drainage tubing that connects to the drainage bag. At 750, the method may further include closing the bladder management device, wherein the decrease of pressure caused by the complete influx of drainage fluid from the user allows the re-attraction between the at least two magnets to move the at least one slidable or articulating or fully movable magnet moves towards the at least one fixed magnet. In some aspects, the closing of the bladder management device may indicate that a user's fluids have been completely and successfully drained, emptying the bladder of all fluid. This would herald the completion of the bladder emptying cycle to automatically start again, fully self-powered by fluid dynamic forces.

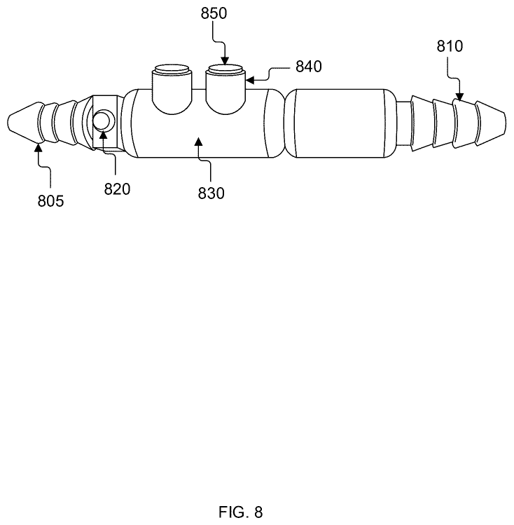

Referring now to FIG. 8, a bladder management device with one or more diagnostic ports, according to some embodiments of the present disclosure, is illustrated. In some aspects, the bladder management device may include a hard, durable body with an inflow portion 805 and an outflow portion 810. In some aspects, the hard, durable body may be transparent. In some implementations, the hard, durable body may include a hollow chamber 830. In some embodiments, the hollow chamber 830 may include a combination sample port and over-pressurization port 820 and at least two magnets. In some embodiments, the sample port and over-pressurization port 820 may be located separately, wherein a sample port and an over-pressurization port are two different accessories to the bladder management device.

In some embodiments, the bladder management device may include one or more diagnostic ports 840 attached to the exterior of the hollow chamber 830, wherein the one or more diagnostic ports 840 may protrude from the exterior surface of the hollow chamber 830. In some implementations, the one or more diagnostic ports 840 may be configured to enable the withdrawal of drainage fluids that enter through the inflow portion 805, wherein the one or more diagnostic ports 840 may be sterilely attached to allow the reentry of sterile drainage fluids back into the hollow chamber 830. In some aspects, the one or more diagnostic ports 840 may include one or more diagnostic caps 850, wherein the one or more diagnostic caps 850 may be detachably connected to the one or more diagnostic ports 840.

In some implementations, the one or more diagnostic caps 850 may be configured to seal off and block a pathway within one or more modular diagnostic ports. In some aspects, the one or more diagnostic caps 850 may be sterilely configured. In some embodiments, the one or more diagnostic caps 850 may be configured to attach to the one or more diagnostic ports 840, wherein the removal of the one or more diagnostic caps 850 may provide access to the drainage fluids through the one or more diagnostic ports 840. By way of example and not limitation, the one or more diagnostic caps 850 may be configured to attach and seal the one or more diagnostic ports 840, wherein the one or more diagnostic caps 850 may press fit, elastically fit, or be locked into the one or more diagnostic ports 840. In some aspects, the one or more diagnostic caps 850 may include a twisting lock, wherein the one or more diagnostic caps 850 may include a threaded exterior that twists and locks into a threaded interior of the one or more diagnostic ports 840, as a non-limiting example.

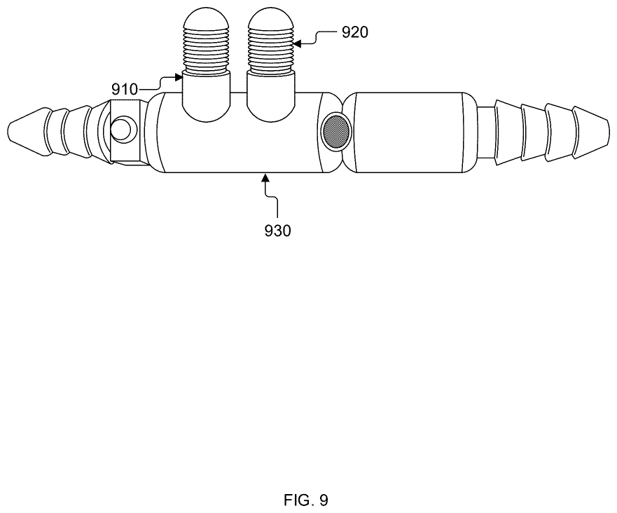

Referring now to FIG. 9, a bladder management device with one or more modular diagnostic pods, according to some embodiments of the present disclosure, is illustrated. In some embodiments, the bladder management device may include one or more diagnostic ports 910 and one or more modular diagnostic pods 920. In some implementations, the one or more diagnostic ports 910 may be included on the hollow chamber 930 of the bladder management device. In some aspects, the bladder management device the one or more modular diagnostic pods 920 may be detachably connectable to the one or more diagnostic ports 910.

In some embodiments, the one or more diagnostic ports 910 may include one or more diagnostic caps, wherein the one or more diagnostic caps may prevent access into the hollow chamber 930 of the bladder management device. In some implementations, the one or more diagnostic caps may be removed from the one or more diagnostic ports 910 to allow connection of the one or more modular diagnostic pods 920 to the one or more diagnostic ports 910. By way of example and not limitation, the one or more modular diagnostic pods 910 may be attached temporarily or permanently to the one or more diagnostic ports 910. In some aspects, the one or more modular diagnostic pods may be sterile and disposable, as non-limiting examples.

In some aspects, the one or more modular diagnostic pods 920 may be configured to access fluids in the hollow chamber of the bladder management device through its connection to the one or more diagnostic ports 910. In some implementations, the one or more modular diagnostic pods 920 may include a point of care digitally reporting microfluidic chip, a point of care digitally reporting microchip, an ultrasound-laser-microchip to measure fluid pressure and volume, a visual chemistry panel card, or a vacuum, as non-limiting examples. The modular diagnostic pods may work independently or in concert with another diagnostic pod. In some embodiments, the microfluidic chip, the visual chemistry panel card, or the vacuum may be configured to draw sterile, fresh fluids through the one or more diagnostic ports 910 and into the one or more modular diagnostic pods 920 for evaluation. In some aspects, the fluids may include urine, as a non-limiting example.

In some embodiments, the one or more modular diagnostic pods 920 may be electronically connectable to a battery source of the bladder management device. In some aspects, the one or more diagnostic ports 910 may include a power mechanism, wherein the one or more diagnostic ports 910 may be electronically coupled to the battery source, wherein the power mechanism may be configured to further connect the battery source to the one or more modular diagnostic pods 920. In some aspects, the power mechanism may be configured to provide electricity to the one or more modular diagnostic pods 920, wherein the one or more modular diagnostic pods 920 may require electricity to perform its function.

By way of example and not limitation, the one or more modular diagnostic pods 920 may include the microfluidic chip or stimulus responsive wireless sensor and transmitter lab on a chip device for measuring microdroplets. In some implementations, the microfluidic chip may be configured to collect low volumes of fluids for chemical analysis and diagnostics, as non-limiting examples. In some aspects, the microfluidic chip may include a tiny system of channels and chambers that may guide fluids through its various components, wherein the microfluidic chip may be configured to evaluate and monitor the fluids. In some aspects, the microfluidic chip may be configured to mix, separate, and detect compounds within the fluid.

By way of example and not limitation, the one or more modular diagnostic pods 920 may include the visual chemistry panel card. In some embodiments, the visual chemistry panel may be configured to provide sterile urine visual testing results. In some aspects, the visual chemistry panel may include panels of chemical tests configured to provide visual indication of the status and condition of the fluids. By way of example and not limitation, the visual chemistry panel card may include tests for diabetes, metabolic, drug detection, and enzymes. In some implementations, the one or more modular diagnostic pods 920 with the visual chemistry panel card may be removed once the fluids may be received by the visual chemistry panel card, wherein the one or more modular diagnostic pods 920 may provide visibility of the visual chemistry panel card to view the results. In some aspects, the results of the visual chemistry panel card may be uploaded to a patient's EHR or point of care external reading and digital reporting device, wherein the results may be viewable immediately as digitally reported to the HER monitoring dashboard.

By way of example and not limitation, the one or more modular diagnostic pods 920 may include the vacuum. In some aspects, the vacuum may be configured to draw fluids into the one or more modular diagnostic pods 920, wherein the fluids may be subject to chemical analysis and diagnostics. In some implementations, the vacuum may be configured to draw in a predetermined quantity of fluid.

In some embodiments, the bladder management device may be electronic, wherein the bladder management device includes the battery source and a microchip. In some implementations, the battery source may provide electricity to the microchip and a connected device like the one or more modular diagnostic pods 920, as a non-limiting example. In some aspects, the one or more modular diagnostic pods 920 may send and receive signals from the microchip of the bladder management device. In some aspects, the one or more modular diagnostic pods 920 and the bladder management device may send and receive signals from an external device or platform, as a non-limiting examples.

In some implementations, the one or more modular diagnostic pods 920 and the bladder management device may be configured to send substantially real-time data to the external or device or platform. By way of example and not limitation, the one or more modular diagnostic pods 920 and the bladder management device may report the results directly to a user's electronic health record. In some aspects, the one or more modular diagnostic pods 920 and the bladder management device may be electronically coupled to the external device, wherein the status and condition of the bladder management device and a chemical analysis and diagnostics of the fluids may be displayed on an external monitor. In some implementations, the external monitor may be connected to the external device, wherein the external device may include the platform, wherein the platform may include a software platform, as a non-limiting example.

In some implementations, the one or more modular pods 920 may include microchip technology interfacing in real time with artificial intelligence (AI) software, wherein the AI software may be configured to receive and analyze signals from the bladder management device and the one or more modular pods 920. In some embodiments, the AI software may be configured to control the one or more modular pods 920, wherein the chemical testing and diagnostics of the fluids may be adjusted or initiated by the AI software. In some aspects, the AI software may automatically activate the one or more modular diagnostics pods 920, wherein the AI software may prompt the one or more modular pods 920 to draw or analyze the fluids, for point of care reporting in real time or for immediate visual test analysis. In some aspects, the AI software may receive the data reported by the one or more modular pods 920, wherein the AI software may analyze and draw conclusions from the results of the one or more modular pods 920. In some aspects, the AI software may be part of a software platform configured to organize and display the results of the one or more modular diagnostics pods 920.

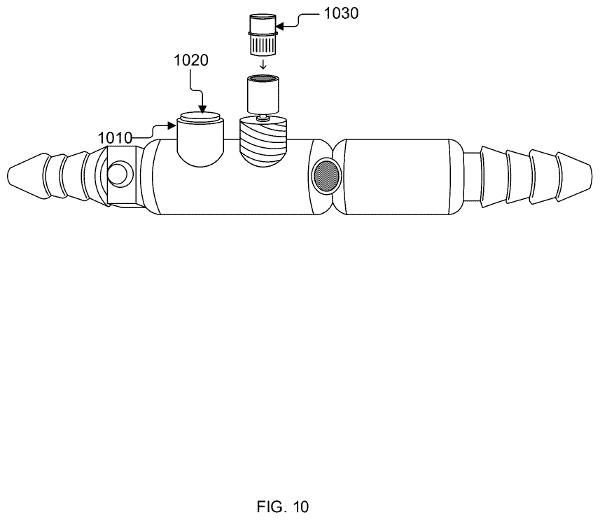

Referring now to FIG. 10, a bladder management device with modular diagnostic pods, according to some embodiments of the present disclosure, is illustrated. In some embodiments, the bladder management device may include one or more diagnostic ports 1010, one or more diagnostic caps 1020, and one or more diagnostic pods. In some implementations, the one or more diagnostic ports 1010 may be configured to receive either one or more diagnostic caps 1020 or one or more diagnostic pods, wherein each of the one or more diagnostic ports 1010 may be configured to receive one or more diagnostic caps 1020 or one or more diagnostic pods 1030.

In some embodiments, the one or more diagnostic ports 1010 may include a receiving mechanism, wherein the receiving mechanism may be configured to connect to a connecting mechanism of the one or more diagnostic caps 1020 or the one or more diagnostic pods. In some implementations, the one or more diagnostic caps 1020 or the one or more diagnostic pods may be detachably connected to the one or more diagnostic ports 1010. By way of example and not limitation, the connecting mechanism may attach to the receiving mechanism through snap fit, press fit, knob and twist fit, or a Luer Lock.

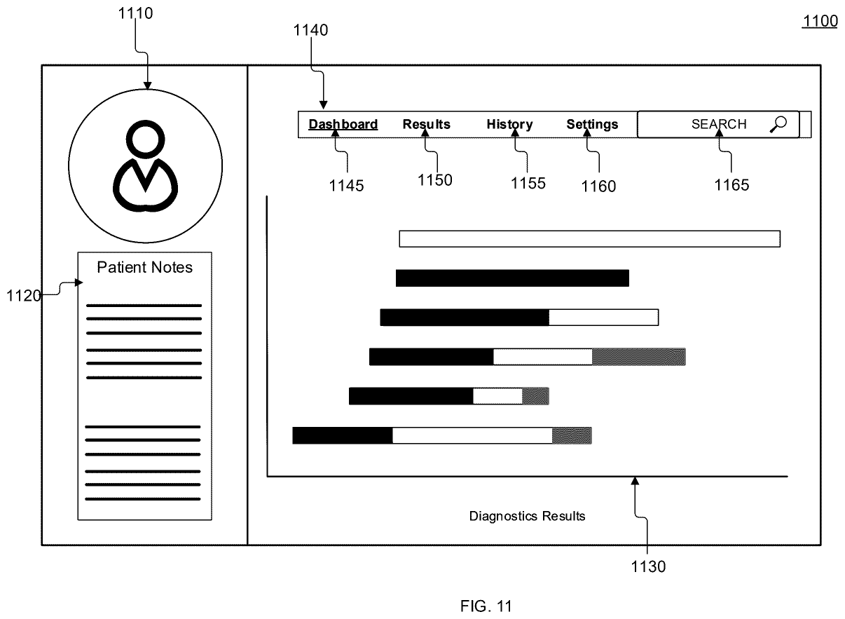

Referring now to FIG. 11, a bladder management system with a bladder management platform, according to some embodiments of the present disclosure, is illustrated. In some embodiments, the bladder management platform 1100 may include a software application electronically coupled to a bladder management device, wherein the bladder management platform 1100 may send and receive signals to the bladder management device. In some aspects, the bladder management device may include a microchip or stimulus responsive wireless sensor and transmitter lab on a chip device for measuring microdroplets, or one or more diagnostic pods configured to send and receive signals to an external device. In some aspects, the external device may include the bladder management platform, a mobile application or a centralized patient EHR (electronic health record), wherein data sent from the bladder management device may be uploaded to the bladder management platform.

In some implementations, the bladder management device may send real-time data to the external device. By way of example and not limitation, the data sent by the bladder management device may include chemical analysis of fluids, diagnostic analysis of fluids, information regarding whether the bladder management device may be working correctly, and information regarding the amount and nature of the received fluid. In some embodiments, the bladder management device may send the data directly to a patient's electronic health record (EHR), as a non-limiting example. In some implementations, the bladder management platform 1100 may be configured to receive information from the patient's EHR, wherein the bladder management platform 1100 may receive information from the bladder management device from its access and information received from the patient's EHR.

In some embodiments, the bladder management platform 1100 may be configured to display, organize, control, improve, and interpret, as non-limiting examples, the results from the bladder management device. In some implementations, the bladder management platform 1100 may include a user interface, wherein the user interface may include a patient profile identifier 1110, patient notes 1120, diagnostic results 1130, and a navigation panel 1140. In some aspects, the patient profile identifier 1110 may include an image, photo, name, or identifier for a patient using the bladder management device, as non-limiting examples.

In some embodiments, the patient notes 1120 may be configured to provide an area on the user interface where a user may type text that may be displayed on the user interface, wherein the text may be directed to observations and details noted by the user. In some aspects, the navigation panel 1140 may include options for navigating the bladder management platform 1100, wherein selecting one of the options may navigate a user to a different page, display, or section of the bladder management platform 1100, as a non-limiting example. By way of example and not limitation, the navigation panel 1140 may include a dashboard option 1145, a results option 1150, a history option 1155, a settings option 1160, and a search option 1165, as non-limiting examples.

In some implementations, the dashboard option 1145 may be configured to display an overview of the bladder management platform. In some aspects, the results option 1150 may be configured to display the results of the bladder management device, wherein the results option 1150 may organize and interpret the data received by the bladder management device. In some implementations, the results option 1150 may be configured to draw inferences and conclusions regarding the fluids analyzed by one or more modular diagnostics pods on the bladder management device. In some implementations, the history option 1155 may be configured to display the patient's history using the bladder management device. In some aspects, the history option 1155 may track the patient's usage of the bladder management device and the status of their fluids. In some aspects, the search option 1165 may be configured to include a search box, wherein the user may type characters in the search box to search for specific terms or words that may be included on the bladder management platform 1100. In some aspects, the typing of specific terms or words may navigate the user to a desired location on the bladder management platform 1100. By way of example and not limitation, the user may type “RESULTS”, wherein the bladder management platform may navigate the user to the results option 1150.

In some embodiments, the bladder management platform 1100 may include the diagnostic results 1130. In some implementations, the diagnostic results 1130 may be configured to display information received by the bladder management device. In some aspects, the bladder management device may be configured to drain and then physiologically cycle the influx of fluids from the patient and act as an automatic self-energizing external prosthetic urinary sphincter. In some implementations, the bladder management device may be configured to act as an automatic self-energizing explantable or implantable urinary sphincter. In some implementations, the bladder management device may include the one or more modular diagnostic pods configured to chemically and diagnostically test the fluids received by the patient. In some aspects, the bladder management platform may be configured to receive the data from the bladder management device regarding the fluid and its chemical and diagnostic properties. In some implementations, the bladder management platform 1100 may display this information in the diagnostic results 1130. By way of example and not limitation, the diagnostic results 1130 may include a determination by the user or the bladder management platform 1110 that the fluids received from a patient indicate a certain condition or status. In some aspects, the bladder management platform 1100 may be configured to display a variety of graphical displays regarding the information received from the bladder management device.

In some embodiments, the bladder management platform 1100 may be configured to send signals to the bladder management device and the one or more modular diagnostic pods, wherein the user of the bladder management platform 1100 may control the functionality of the bladder management device and the one or more diagnostic pods. In some aspects, the bladder management platform 1100 may be configured to adjust the settings of the bladder management device 1100 or calibrate the bladder management device 1100 to the patient, as non-limiting examples. In some implementations, the bladder management platform 1100 may be configured to allow the user to select which chemical or diagnostic tests may be conducted by the one or more modular diagnostic pods.

In some embodiments, the bladder management platform 1100 may include artificial intelligence (AI) software. By way of example and not limitation, the AI software of the bladder management platform 1100 may be configured to automate the functionality of the bladder management device and the testing of the one or more modular diagnostic pods. In some aspects, the AI software may be configured to automatically interpret, organize, and improve the bladder management platform 1100. In some embodiments, the AI software may generate the results and organize the display on the bladder management platform 1100. In some aspects, the AI software may interpret the data received and generate conclusions regarding the chemical properties of the fluid.

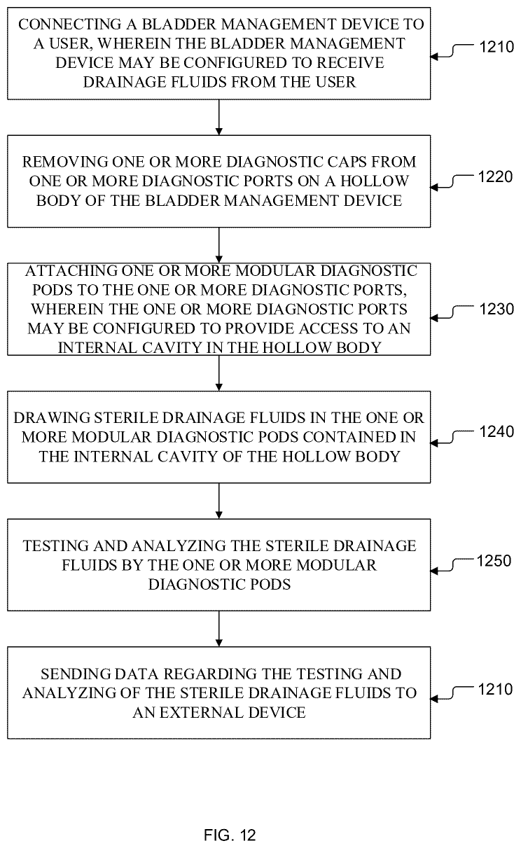

Referring now to FIG. 12, a method for bladder management, according to some embodiments of the present disclosure, is illustrated. At 1210, connecting a bladder management device to a user, wherein the bladder management device may be configured to receive drainage fluids from the user. At 1220, removing one or more diagnostic caps from one or more diagnostic ports on a hollow body of the bladder management device. In some aspects, the one or more diagnostic caps may be locked into the one or more diagnostic ports, wherein the one or more diagnostic caps may be unlocked before they may be removed from the one or more diagnostic ports. At 1230, attaching one or more modular diagnostic pods to the one or more diagnostic ports, wherein the one or more diagnostic ports may be configured to provide access to an internal cavity in the hollow body. In some aspects, the one or more modular diagnostic pods may be locked or securely attached to the diagnostic ports, as non-limiting examples.

At 1240, drawing sterile drainage fluids in the one or more modular diagnostic pods contained in the internal cavity of the hollow body. In some aspects, the drainage fluids may be urine, as a non-limiting example. In some aspects, the drawing of the sterile urine may be no-touch, wherein the sterile drainage fluids may be configured to move in and out of the one or more diagnostic pods without any contamination, as a non-limiting example. At 1250, testing and analyzing the sterile drainage fluids by the one or more modular diagnostic pods. In some aspects, the one or more modular diagnostic pods may analyze the chemistry, cell, bacteria, pH, or urine metric data, as non-limiting examples.

At 1260, sending data regarding the testing and analyzing of the sterile drainage fluids to an external device. In some aspects, the external device may include a patient's electronic health record (EHR) or a bladder management platform. In some embodiments, the bladder management platform may be configured to send and receive data from the bladder management device, wherein the bladder management platform may provide a user interface and graphical displays to review and visualize the data received from the one or more modular diagnostic pods.

### CONCLUSION

A number of embodiments of the present disclosure have been described. While this specification contains many specific implementation details, these should not be construed as limitations on the scope of any disclosures or of what may be claimed, but rather as descriptions of features specific to particular embodiments of the present disclosure.

Certain features that are described in this specification in the context of separate embodiments can also be implemented in combination or in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Moreover, the separation of various system components in the embodiments described above should not be understood as requiring such separation in all embodiments, and it should be understood that the described components and systems can generally be integrated together in a single product or packaged into multiple products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order show, or sequential order, to achieve desirable results. In certain implementations, multitasking and parallel processing may be advantageous. Nevertheless, it will be understood that various modifications may be made without departing from the spirit and scope of the claimed disclosure.

Reference in this specification to “one embodiment,” “an embodiment,” any other phrase mentioning the word “embodiment”, “aspect”, or “implementation” means that a particular feature, structure, or characteristic described in connection with the embodiment is included in at least one embodiment of the disclosure and also means that any particular feature, structure, or characteristic described in connection with one embodiment can be included in any embodiment or can be omitted or excluded from any embodiment. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others and may be omitted from any embodiment. Furthermore, any particular feature, structure, or characteristic described herein may be optional.

Similarly, various requirements are described which may be requirements for some embodiments, but not other embodiments. Where appropriate any of the features discussed herein in relation to one aspect or embodiment of the invention may be applied to another aspect or embodiment of the invention. Similarly, where appropriate any of the features discussed herein in relation to one aspect or embodiment of the invention may be optional with respect to and/or omitted from that aspect or embodiment of the invention or any other aspect or embodiment of the invention discussed or disclosed herein.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks: The use of highlighting has no influence on the scope and meaning of a term; the scope and meaning of a term is the same, in the same context, whether or not it is highlighted.

It will be appreciated that the same thing can be said in more than one way. Consequently, alternative language and synonyms may be used for any one or more of the terms discussed herein. No special significance is to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

It will be appreciated that terms such as “front,” “back,” “top,” “bottom,” “side,” “short,” “long,” “up,” “down,” “aft,” “forward,” “inboard,” “outboard,” “upstream,” “downstream,” “indwelling,” “EHR”, and “below” used herein are merely for ease of description and refer to the orientation of the components as shown in the figures. It should be understood that any orientation of the components described herein is within the scope of the present invention.

What is claimed is:

1. A bladder management device comprising:
a hard, durable body with a hollow chamber including:
an inflow portion, wherein the inflow portion is configured to accept the influx of drainage fluids from at least one user;
an outflow portion is comprised of distally oriented barbed fins, wherein the outflow portion is configured to expel fluids to at least one external source;
a sample port configured to enable the hollow chamber to collect a sterile fluid specimen;
an over-pressurization port safety relief valve to ensure failsafe release of excess pressure in the hollow chamber;
at least two magnets including at least one fixed magnet and at least one slidable, articulating, or fully movable magnet, wherein the at least one slidable, articulating, or fully moveable magnet moves when a certain predetermined threshold of pressure is experienced;
one or more diagnostic ports protruding from the exterior surface of the hollow chamber, wherein the one or more diagnostic ports include a pathway into the hollow chamber; and
one or more modular diagnostic pods detachably connectable to the one or more diagnostic ports, wherein the one or more modular diagnostic pods are configured to draw in drainage fluids from the hollow chamber, wherein the one or more modular diagnostic pods are configured to either perform chemical and diagnostic testing on the drainage fluids before releasing the drainage fluids back into the hollow chamber or to collect the drainage fluids for removal to conduct ex-vivo analysis.

2. The bladder management device of claim 1, wherein the bladder management device further includes one or more diagnostic caps detachably connectable to the one or more diagnostic ports, wherein the one or more diagnostic caps are configured to seal off and block the pathway into the hollow chamber from the one or more diagnostic ports when the one or more modular diagnostic pods are not attached.

3. The bladder management device of claim 1, wherein the one or more diagnostic ports include a receiving mechanism configured to connect to a connecting mechanism on the one or more modular diagnostic pods, wherein the one or more modular diagnostic pods securely attach to the one or more diagnostic ports.

4. The bladder management device of claim 1, wherein the bladder management device further includes a battery source configured to provide electricity to the one or more diagnostic modular diagnostic pods.

5. The bladder management device of claim 1, wherein the one or more modular diagnostic pods include a microfluidic chip or a stimulus responsive wireless sensor and transmitter lab on a chip device configured to collect low volumes of drainage fluids for chemical and diagnostic testing, wherein the microfluidic chip is configured to mix, separate, and detect compounds within the drainage fluid.

6. The bladder management device of claim 1, wherein the one or more modular diagnostic pods include a visual chemistry panel card, wherein the visual chemistry panel card is configured to provide visual indication of the chemical and diagnostic status of the drainage fluids.

7. The bladder management device of claim 1, wherein the one or more diagnostic pods include a vacuum configured to draw fluids from the hollow chamber into the one or more diagnostic pods for chemical testing and diagnostic testing.

8. The bladder management device of claim 1, wherein the one or more diagnostic pods send and receive signals from an external device.

9. The bladder management device of claim 8, wherein the external device includes the at least one user's electronic health record (EHR), wherein the chemical and diagnostic testing are received by the at least one user's electronic health record (EHR).

10. The bladder management device of claim 8, wherein the results of the chemical and diagnostic testing are sent to the external device in substantially real-time.

11. The bladder management device of claim 8, wherein the external device is configured to send signals that control and adjust the functionality of the one or more modular diagnostic pods.

12. A bladder management system including:
a bladder management platform configured to:
send and receive data from one or more modular diagnostic pods connected to a bladder management device, wherein one or more modular diagnostic pods are configured to chemically and diagnostically test fluids in the bladder management device;
upload the results of the chemical and diagnostic testing of the fluids onto a user interface of the bladder management platform, wherein the results are organizable and navigable; and
displaying the results of the chemical and diagnostic testing of the fluids on the user interface, wherein the bladder management platform includes graphical displays of the results.

13. The bladder management system of claim 12, wherein the bladder management platform include artificial intelligence (AI) software, wherein the AI software is configured to automate the sending and receiving of signals to and from the one or more modular diagnostic pods and the bladder management platform.

14. The bladder management system of claim 13, wherein the AI software is configured to organize the results of the one or more modular diagnostic pods and generate the graphical displays for the bladder management platform.

15. The bladder management system of claim 12, wherein the user interface includes options for navigating the bladder management platform.

16. A method for a bladder management including:
connecting a bladder management device to a user, wherein the bladder management device is configured to receive drainage fluids from the user;
removing one or more diagnostic caps from one or more diagnostic ports on a hollow body of the bladder management device;
attaching one or more modular diagnostic pods to the one or more diagnostic ports, wherein the one or more diagnostic ports provide access to an internal cavity in the hollow body;
drawing sterile drainage fluids into the one or more modular diagnostic pods contained in the internal cavity of the hollow body;
testing and analyzing the sterile drainage fluids by the one or more modular diagnostic pods; and
sending data regarding the testing and analyzing of the sterile drainage fluids to an external device.

17. The method of claim 16, wherein the drainage fluid is urine.

18. The method of claim 16, wherein the external device includes a user's electronic health record (EHR).

19. The method of claim 16, further including releasing the sterile drainage fluid back into the internal cavity of the hollow chamber by the one or more modular diagnostic pods.

20. The method of claim 16, further including removing the one or more modular diagnostic pods after the testing and analyzing of the sterile drainage fluids, wherein the one or more diagnostic caps are reattached to the one or more diagnostic ports to block access into the hollow chamber.
